(19) United States
(12) Patent Application Publication
(10) Pub. No.: US 2024/0269435 A1
(43) Pub. Date: Aug. 15, 2024

(54) Medical Device Centering Device

(71) Applicant: Bard Peripheral Vascular, Inc., Franklin Lakes, NJ (US)

(72) Inventor: Nathan Gooch, Centerville, UT (US)

(21) Appl. No.: 18/289,544

(22) PCT Filed: May 7, 2021
(86) PCT No.: PCT/US21/31403

§ 371 Date: Nov. 3, 2023

(51) Int. Cl. A61M 25/04

(52) U.S. Cl. CPC A61M 25/04

(57) ABSTRACT

Embodiments of medical device centering systems and associated methods thereof are described herein and include an elongate medical device, such as a catheter, and a centering device configured to transition between a retracted configuration and an extended configuration. In the extended configuration the centering device can be configured to prevent a distal tip of the catheter from contacting a wall of the vessel. The centering device can provide a spaced apart relationship, or center the tip within the vessel. In an embodiment, the centering device can distend the vessel to provide a fluid pathway between the catheter tip and the vessel wall. Embodiments described herein can mitigate arterial insufficiency while allowing a catheter to be fluid locked along an entire length of the lumen.

### SUMMARY

Briefly summarized, embodiments disclosed herein are directed to a centering device for a medical device, configured to position a distal tip of a catheter, or similar elongate medical device, within a vascular and to mitigate arterial insufficiency. When accessing a vasculature with a catheter or similar medical device, the cross-sectional area of the vessel can be reduced by the presence of the device. As such, blood flow through the vessel can be affected. This can be of particular importance when the catheter is used to aspirate fluids from the vasculature such as during dialysis. To mitigate these effects, some catheter devices have been developed to include catheter side apertures extending through a wall of the catheter, disposed adjacent the distal tip, and communicating with the catheter lumen. These side apertures provide additional fluid flow upstream of the distal opening.

However, the side apertures can prevent the entire length of the catheter from being “fluid locked.” Fluid locking can include maintaining a saline solution column along the length of the catheter lumen. Optionally the saline solution can include active ingredients such as anti-thrombus or anti-bacterial agents, heparin, or the like. The fluid lock can prevent the formation of biofilms or the like, which can lead to clotting, thromboses, or infection. The presence of catheter side apertures can prevent the distal portion of the catheter lumen from forming the fluid lock leading to the formation of thromboses and the like.

Embodiments disclosed herein are directed to a centering device configured to maintain a spaced apart relationship between a distal tip of the medical device and the wall of the vessel. In an embodiment, the centering device can maintain the tip of the medical device in a substantially central position. In an embodiment, the vascular centering device can stretch a wall of the vessel to allow a fluid flow to pass the distal tip and mitigate arterial insufficiency. In an embodiment the centering device can align an axis of a portion of the catheter with an axis of the vessel.

Disclosed herein is a centering device configured to access a vasculature of a patient including, a catheter including an elongate body defining a lumen communicating with an opening disposed adjacent a distal tip thereof, and a centering device transitionable between a retracted configuration and an extended configuration, the centering device in the extended configuration configured to distance a distal tip of the catheter from a wall of the vasculature, the centering device including a wire extending longitudinally along an outer surface of the catheter and slidably engaged therewith, the wire having a distal tip coupled to a distal portion of the catheter, an expandable portion disposed proximate the distal tip of the catheter and configured to transition between the retracted configuration and the extended configuration, and a proximal portion configured to remain external to the patient.

In some embodiments, the wire includes one of a shape memory material or nitinol. In some embodiments, the expandable portion is elastically deformable from the retracted configuration to the extended configuration. In some embodiments, the expandable portion includes a hinge or a living hinge. In some embodiments, the proximal portion is slidable along a longitudinal axis between the retracted configuration and the extended configuration. In some embodiments, a portion of the wire is disposed within a groove defined in an out surface of the catheter. In some embodiments, the centering device further includes the expandable portion of the wire disposed within the groove when the wire is in the retracted configuration. In some embodiments, the centering device further includes the proximal portion of the wire disposed within the groove and slidably engaged therewith.

In some embodiments, a radial depth of the groove is equal to a thickness of the wire. In some embodiments, the centering device further includes a bridge formed integrally with the catheter body and extending across the groove, perpendicular thereto, the bridge configured to retain a portion of the wire within the groove. In some embodiments, a portion of the wire extends through a wire lumen extending through a wall of the catheter. In some embodiments, the centering device further includes a loop formed integrally with the catheter body and configured to receive a portion of the wire extending therethrough. In some embodiments, a lumen of the loop is angled relative to the longitudinal axis.

In some embodiments, the centering device further includes an abutment formed integrally with the catheter, the distal tip of the wire configured to abut against the abutment and prevent further distal movement of the distal tip relative to the catheter. In some embodiments, the proximal portion of the wire is coupled to a second proximal portion of a second wire to form a loop, the loop configured to be advanced distally to transition one of the wire or the second wire between the retracted configuration and the extended configuration. In some embodiments, the centering device further includes a push-button actuating mechanism configured to transition the wire between the retracted configuration and the extended configuration.

In some embodiments, an apex of the expandable portion in the extended configuration is disposed radially outward from a central longitudinal axis relative to the retracted configuration. In some embodiments, an apex is disposed a first distance from the distal tip, the first distance being less than three times the diameter of the lumen. In some embodiments, the distal tip of the wire is disposed proximal of the distal tip of the catheter. In some embodiments, the distal tip of the wire is disposed a second distance from the distal tip, the second distance being less than two times the diameter of the lumen. In some embodiments, the proximal portion is disposed adjacent a bifurcation of the catheter.

In some embodiments, the expandable portion in the extended configuration is configured to distend a vessel wall to a larger diameter along a first axis extending perpendicular to a longitudinal axis. In some embodiments, the centering device in the extended configuration is configured to prevent a distal tip of the catheter from contacting a wall of the vasculature. In some embodiments, the centering device further includes a second expandable portion disposed axially relative to the expandable portion, the second expandable portion including a second apex contacting a vessel wall at a different longitudinal configuration from the first apex. In some embodiments, the lumen of the catheter is fluid locked between a hub disposed at a proximal end of the elongate body and a distal tip.

Also disclosed is a method of centering a catheter within a vasculature of a patient including, advancing a distal tip of the catheter into the vasculature, the catheter including an elongate body defining a lumen communicating with a distal opening, sliding a proximal portion of a wire longitudinally distally, the proximal portion slidably engaged with an outer surface of the elongate body, a distal tip of the wire coupled to the catheter and proximal of the distal tip of the catheter, and the proximal portion configured to remain proximal of an insertion site, and transitioning an articulated portion of the wire between a retracted position and an extended position, the articulated portion disposed adjacent the distal tip of the catheter and configured to prevent the distal tip from contacting a wall of the vasculature when in the extended position.

In some embodiments, the articulated portion is elastically deformable from the retracted position to the extended position. In some embodiments, the articulated portion includes a hinge or a living hinge. In some embodiments, the proximal portion is slidably engaged with a wall of the catheter. In some embodiments, the proximal portion of the wire is coupled to a second proximal portion of a second wire to form a loop, the loop configured to be actuated to transition both the wire and the second wire between the retracted position and the extended position. In some embodiments, the method further includes a push-button actuating mechanism configured to transition the wire between the retracted position and the extended position. In some embodiments, an apex of the articulated portion in the extended position is disposed radially outward from a central longitudinal axis relative to the retracted position. In some embodiments, the proximal portion is disposed adjacent a bifurcation of the catheter. In some embodiments, the wire includes one of a shape memory material or nitinol. In some embodiments, the articulated portion in the extended position is configured to distend a vessel wall to a larger diameter.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a medical device centering system in a retracted configuration, in accordance with embodiments disclosed herein.

FIG. 1B shows a perspective view of a medical device centering system in an extended configuration, in accordance with embodiments disclosed herein.

FIG. 2A shows a longitudinal cross-section view of a medical device centering system in a retracted configuration, in accordance with embodiments disclosed herein.

FIG. 2B shows a longitudinal cross-section view of a medical device centering system in an extended configuration, in accordance with embodiments disclosed herein.

FIG. 2C shows a longitudinal cross-section view of a medical device centering system in a second extended configuration, in accordance with embodiments disclosed herein.

FIG. 2D shows close up detail of a distal loop of the device of FIG. 2B, in accordance with embodiments disclosed herein.

FIG. 3A shows a perspective view of a medical device centering system including an actuator in a retracted configuration, in accordance with embodiments disclosed herein.

FIG. 3B shows a perspective view of a medical device centering system including an actuator in an extended configuration, in accordance with embodiments disclosed herein.

FIG. 3C shows a lateral cross-section view of a medical device centering system, in accordance with embodiments disclosed herein.

FIG. 3D shows a lateral cross-section view of a medical device centering system, in accordance with embodiments disclosed herein.

FIG. 3E shows a longitudinal cross-section view of a medical device centering system, in accordance with embodiments disclosed herein.

FIG. 3F shows a longitudinal cross-section view of a medical device centering system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

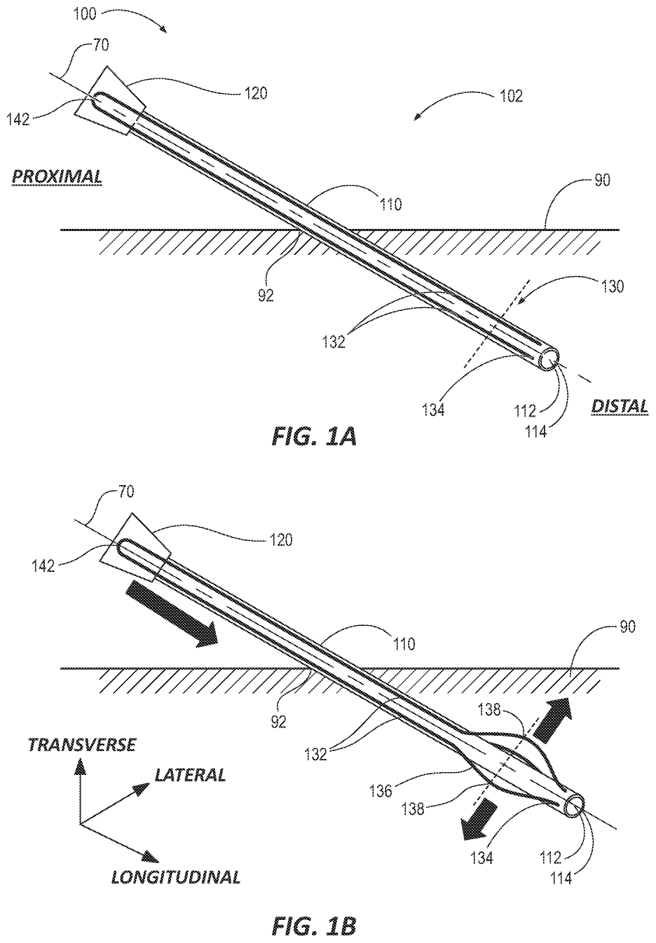

To assist in the description of embodiments described herein, as shown in FIG. 1B, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

Embodiments disclosed herein are directed to medical device centering device configured maintain a spaced apart relationship between a distal tip of the medical device and the wall of the vessel. In an embodiment, the centering device can maintain the tip of the medical device in a substantially central position. In an embodiment, the vascular centering device can stretch the vessel to allow a fluid flow to pass the distal tip.

FIGS. 1A-1B show perspective views of a medical device centering system (“system”) 100 generally including an elongate medical device, or “catheter” 102 and a centering device 130. The catheter 102 can generally include an elongate body 110 extending along a longitudinal axis and defining a lumen 112. The body 110 can be supported by a catheter hub 120 disposed at a proximal end. Optionally, one or more extension legs can extend proximally from the hub 120, each extension leg can be in fluid communication with a lumen 112 of the catheter 102. Optionally, the catheter 102 can include two or more lumen 112, each lumen being in fluid communication with an extension leg. As will be appreciated the catheter 102 is an exemplary medical device and embodiments described herein can be used with various elongate medical devices such as catheters, sacrificial catheters, introducers, or similar devices configured to access a vasculature 80 of a patient.

The system 100 can further include a centering device 130 including a wire 132 extending longitudinal and slidably engaged with the catheter 102 along the longitudinal axis. In an embodiment, the wire 132 can extend along an outer surface of the catheter body 110 and can be slidably engaged therewith. In an embodiment, a portion of the wire 132 can extend through a wall of the catheter body 110 and can be slidably engaged therewith, as described in more detail herein.

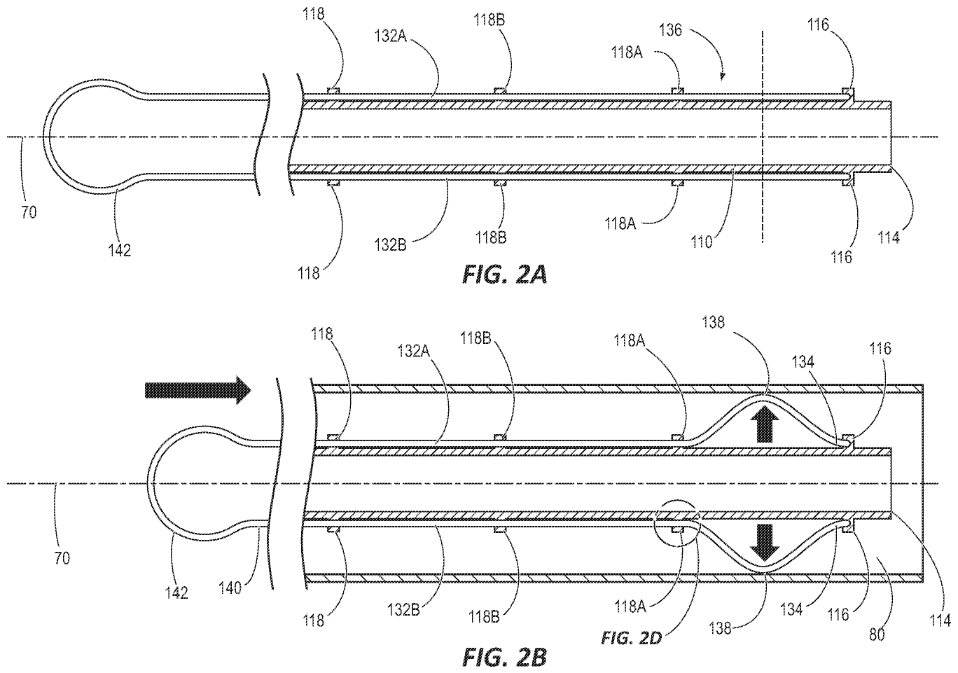

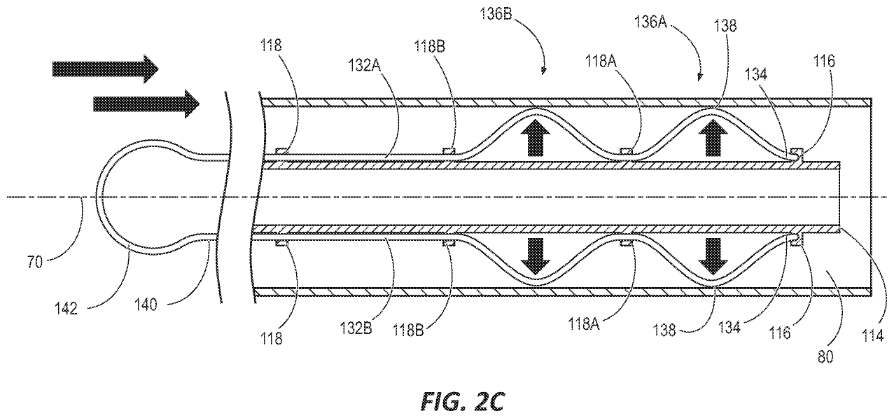

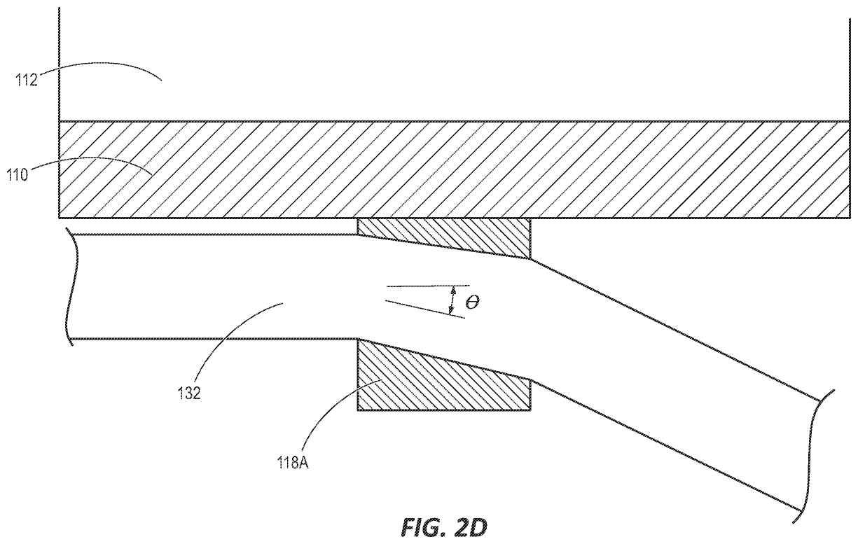

In an embodiment, the wire 132 can include an expandable, or articulated, portion 136 disposed adjacent a distal portion of the wire 132. In an embodiment, as shown in FIGS. 1A-2B, the expandable portion 136 can transition between a retracted configuration (e.g. FIGS. 1A, 2A) and an extended configuration (e.g. FIGS. 1B, 2B). In the retracted configuration the expandable portion 136 can lie flat against an outer surface of the body 102. In an embodiment, in the retracted configuration the expandable portion 136 of the wire 132 can lie parallel with a longitudinal axis of the body 102. In the extended configuration, the expandable portion 136 can extend away from the outer surface of the body 110. In an embodiment, in the extended configuration the expandable portion 136 can define a non-linear or curved profile. In an embodiment, an apex 138 of the expandable portion 136 in the extended configuration can be disposed radially outward from a central longitudinal axis 70 relative to the apex 138 in the retracted configuration. In an embodiment, the apex 138 can contact a wall of the vasculature 80. In an embodiment, the apex 138 of the expandable portion 136 can be disposed at a first distance from the distal tip 114 of the catheter 102.

In an embodiment, a distal tip 134 of the wire 132 can be fixedly engaged with the catheter body 110 to prevent the distal tip 134 from moving distally relative to the body 110. In an embodiment, as shown in FIGS. 2A-2B, the distal tip 134 can abut against an abutment 116, formed integrally with the catheter body 110 and configured to prevent the distal tip 134 from advancing distally relative to the body 110. In an embodiment, the distal tip 134 of the wire 132 can be coupled to the catheter 102 adjacent to the distal tip 114 of the catheter 102. In an embodiment, the distal tip 134 of the wire 132 can be coupled to the catheter proximal of the distal tip 114 of the catheter 102. In an embodiment, a distance (“second distance”) between the distal tip 134 of the wire 132 and a distal tip 114 of the catheter 102 can be equal to, or less than, a distance that is twice the diameter of the lumen 112 of the catheter 102. In an embodiment, the distal tip 134 of the wire 132 can be coupled to the catheter 102 within between 1 mm and 50 mm from the distal tip 114 of the catheter 102. However, it will be appreciated that greater or lesser distances are contemplated.

Advantageously, the distance between one of the distal tip 134 of the wire 132 or the apex 138 of the expandable portion 136 and the distal tip 114 of the catheter can be configured to prevent the catheter tip 114 from contacting the wall of the vessel when the expandable portion 136 is in the extended configuration. Worded differently, one of the distal tip 134 of the wire 132 or the apex 138 of the expandable portion 136 can be disposed within a distance of the catheter distal tip 114 that can prevent the distal tip 114 from contacting a portion of the vessel wall disposed substantially perpendicular thereto.

In an embodiment, the expandable portion 136 in the retracted configuration can be configured to allow a portion of the catheter body 110, longitudinally aligned therewith, to flex. For example, as shown in FIG. 2A the portion of the catheter body 110 disposed longitudinally between the first loop 118A and the abutment 116, i.e. longitudinally aligned with the expandable portion 136, can be configured to flex along an axis extending perpendicular to the longitudinal axis 70.

In an embodiment, the expandable portion 136 in the extended configuration can be configured to maintain a portion of the catheter lumen 112, longitudinally aligned therewith, in a substantially straight configuration, preventing the portion of the body 110 from flexing. In an embodiment, the expandable portion 136 in the extended configuration can be configured to maintain an axis of the catheter lumen 112 aligned parallel with an axis of the vasculature 80, For example, as shown in FIG. 2B the portion of the catheter lumen 112 disposed longitudinally between the first loop 118A and the abutment 116, can be maintained in a straight configuration, or parallel with the axis of the vasculature 80. Advantageously, the expandable portion 136 in the extended configuration can maintain alignment of an axis of the lumen 112 with an axis of the vasculature 80 reducing fluid resistance and improving arterial insufficiency.

In an embodiment, as shown in FIGS. 1A-2B, a proximal portion of the wire 132 can be slidably engaged with the catheter body 110. Sliding the proximal portion of the wire 132 distally with the distal tip 134 fixed attached to the catheter body 110 can cause the expandable portion 136 to transition from the retracted configuration to the extended configuration. Similarly, sliding the proximal portion of the wire 132 proximally can cause the expandable portion 136 to transition from the extended configuration to the retracted configuration.

In an embodiment, a portion of the wire 132 can be slidably engaged with an outer surface of the catheter body 110 and secured in place with one or more loops 118. The loops 118 can be formed integrally with the catheter body 110 or can be formed as a separate structure and coupled to the catheter 102. Advantageously, the centering device 130 can then be coupled with a pre-existing medical device. The loops 118 can be configured to allow a proximal portion of the wire 132 to slide longitudinally.

In an embodiment, as shown in FIG. 2C, the centering device 130 can include a first expandable portion 136A disposed between a first loop 118A and the abutment 116, and disposed at a distal end of the catheter body 110. In an embodiment, the centering device 130 can include a second expandable portion 136B disposed longitudinally proximally of the first expandable portion 136A, for example between a second loop 118B and the first loop 118A, where the second loop 118B is disposed proximally of the first loop 118A. In an embodiment, the system 100 can include two or more expandable portions 136A, 136B aligned axially along the catheter 102. In an embodiment, the expandable portion(s) 136 can maintain an axis of the catheter lumen 112 in a straight configuration and/or align a portion of the catheter lumen 112, e.g. a portion of the catheter longitudinally aligned with the expandable portion 136, with an axis of the vasculature 80. Advantageously, the expandable portion can align an axis of the catheter, and as such an axis of fluid flow therethrough, with an axis of the vasculature 80 to minimize fluid resistance and mitigate arterial insufficiency.

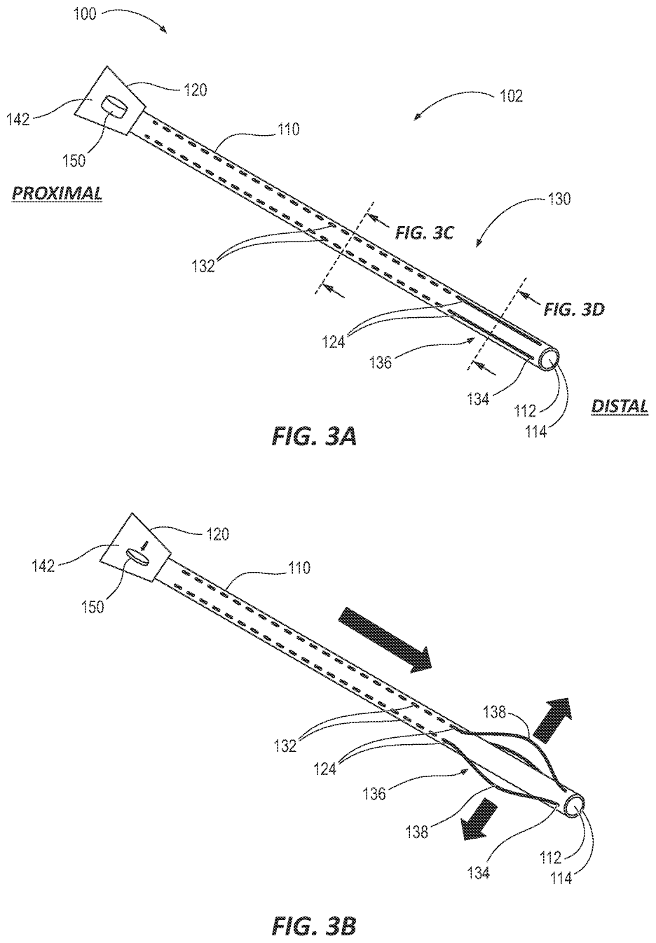

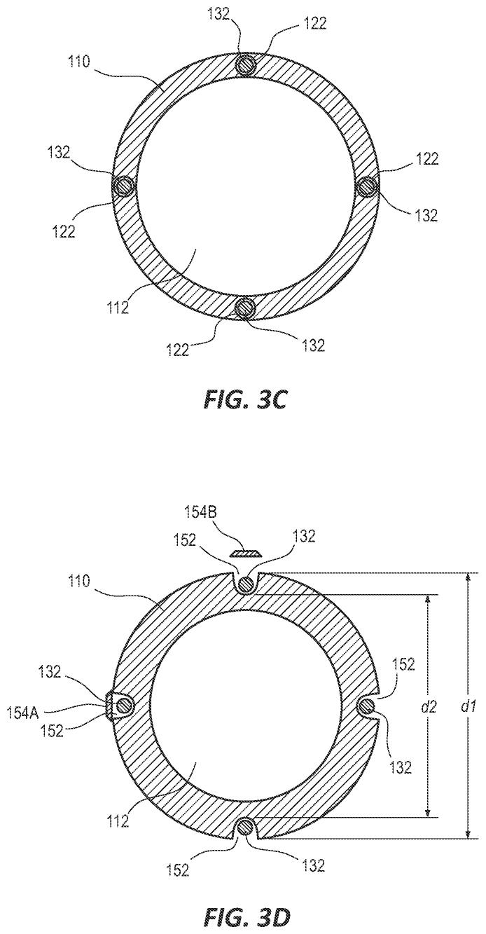

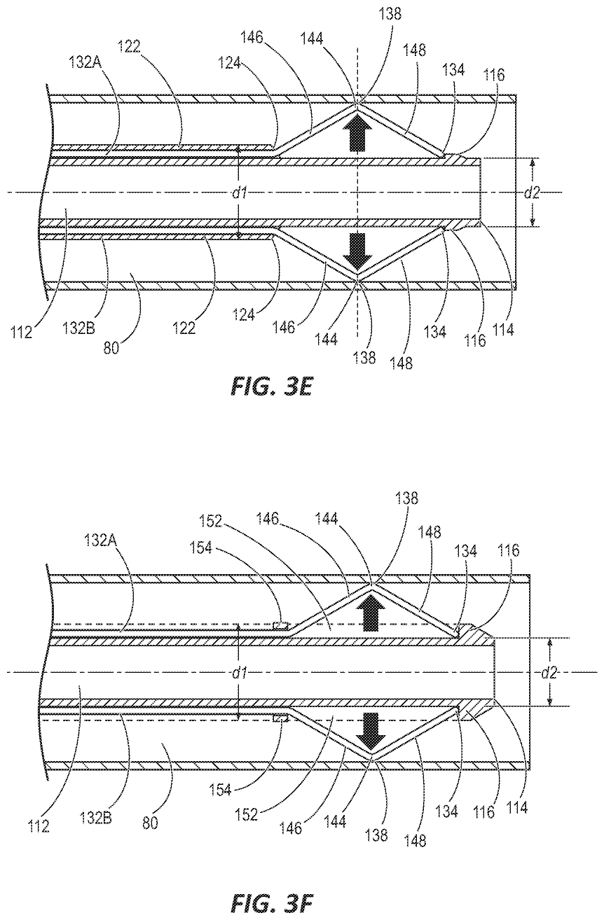

In an embodiment, as shown in FIGS. 3A-3C, 3E, a portion of the wire 132 can extend through a wall of the catheter 102. The wall of the catheter 102 can include a wire lumen 122 extending therethrough and configured to receive a portion of the wire 132 slidably engaged therewith. The wire lumen 122 can communicate with an exit aperture 124 disposed in a side wall of the catheter body 102 and communicating with an outer surface of the catheter. In an embodiment, the system 100 can include a loop 118 disposed distally of the exit aperture 124 and configured to receive the wire 132 therethrough. As such, the system 100 can include a first expandable portion 136A disposed between the loop 118 and the distal tip 114 of the catheter 102 and a second expandable portion 136B disposed between the exit aperture 124 and the loop 118. Advantageously, the two or more expandable portions 136, aligned axially, can provide two or more contact points between the system 100 and the wall of the vasculature 80. These two or more contact points can align the axis of the catheter lumen 112, or can distribute pressure applied to the vasculature. Further, the two or more axially aligned contact points can maintain a portion of the vessel wall, disposed therebetween, in a substantially parallel alignment with an axis of the lumen of the vessel 80, or an axis of the lumen 112 of the catheter 102. This can provide increased arterial flow along an axial portion of the vessel 80 reducing fluid resistance.

In an embodiment, as shown in FIGS. 3E-3F, an apex 138 can include a hinge 144 or living hinge to facilitate the transition of the expandable portion 138 between the retracted configuration and the extended configuration. Advantageously, the expandable portion 136 including the hinge 144 can include a first arm 146 and a second arm 148, disposed either side of the hinge 144. Each of the first arm 146 and the second arm 148 can define a linear profile. The linear profile can provide increased strength or resistance to distortion when engaging a side wall of the vessel 80.

In an embodiment, a portion of the wire 132 can include a metal, alloy, plastic, polymer, or composite material. In an embodiment, a portion of the wire 132, e.g. the expandable portion 136, can include a super-elastic or shape memory material, for example nitinol, or the like. The wire 132 can define a retracted configuration in a first phase, e.g. one of a martensite phase or austenite phase, and can define an extended configuration in a second phase, e.g. one of an austenite or a martensite phase. In an embodiment, a difference in temperature between being disposed externally of the vasculature 80 and internally to the vasculature 80 can trigger a change in phase of the shape memory material and can transition the expandable portion between the retracted configuration and the extended configuration. Advantageously, the super-elastic properties can allow for elastic deformation of the expandable portion 136 without kinking or permanent deformation of the wire 132. Advantageously, the shape-memory properties can allow for automatic triggers (e.g. a change in temperature) of the expandable portion 136.

In an embodiment, as shown in FIGS. 2A-2D the expandable portion 136 of the wire 132 can be disposed between a distal-most loop, e.g. a first loop 118A, and the distal tip 134 of the wire 132. In an embodiment, as shown in FIGS. 2B, 2D, the distal-most loop 118A can define an angled lumen extending therethrough. The angle (e) of the lumen can extend at an angle relative to the longitudinal axis and can be configured to facilitate the expandable portion 136 to transition between the retracted configuration and the extended configuration as the wire 132 is urged distally. In an embodiment, as shown in FIGS. 3E-3F the expandable portion 136 of the wire 132 can be disposed between an exit aperture 124 and distal tip 134 of the wire 132. In an embodiment, an exit aperture 124 can be angled (e) similar to the lumen of the first loop 118A to facilitate the expandable portion 136 to transition between the retracted configuration and the extended configuration.

In an embodiment, as shown in FIG. 3E, an outer diameter of the catheter body 110 can define a first diameter (d1). A portion of the catheter body 110, e.g. at the expandable portion 136, can define a second diameter (d2). The second diameter (d2) can be less than the first diameter (d1). Advantageously, the difference between the diameter of the catheter body 110 (d1) and the diameter of the catheter at the expandable portion 136 (d2) can be substantially the same as the thickness of the wire(s) 132 of the expandable portion 136. As such, when the expandable portion is in the retracted position, the outer diameter of the system 100 maintains a substantially uniform outer profile or outer diameter along the length of the device.

In an embodiment, as shown in FIGS. 3D, 3F, a portion of the catheter body 110, e.g. at the expandable portion 136, can include a groove 152 extending longitudinally and configured to receive a portion of the wire 132, e.g. the expandable portion 136 of the wire 132, therein when in the retracted configuration. In an embodiment, the radial depth of the groove 152 can be substantially the same as a thickness of the wire 132. As such, with wire 132 disposed within the groove 152, the wire 132 and the catheter body 110 can maintain a substantially uniform outer profile, or a substantially uniform diameter, e.g. a first diameter (d1). Advantageously, the uniform outer profile can facilitate insertion of the system 100 into the vasculature 80.

In an embodiment, as shown in FIG. 3D, the groove 152 can extend between the distal tip 114 and the catheter hub 120. In an embodiment, the catheter body 110 can include one or more bridges 154 extending across the groove 152 and configured to retain the wire 132 therein. To note, for clarity, FIG. 3D shows a first bridge 154A disposed in position across the groove 152 and a second bridge 154B in an exploded view. The bridges 154 can be formed integrally with the catheter body 110 or can be formed separately and coupled to the catheter body 110 using welding, adhesive, bonding, or the like. Advantageously, the grooves 152 can retain the wire 132 and maintain a substantially uniform outer profile along the length of the catheter body 110. Further, the catheter body 110 including the grooves 152 can provide a more simplified manufacturing process.

In an embodiment, a proximal portion of the wire 132 can extend along an outer surface of the catheter body 110 and can be slidably engaged therewith. In an embodiment, a proximal end of the wire 140 can extend proximally to a point that is proximal to the insertion site 92, when the distal tip 114 is disposed at a target location within the vasculature 80 of the patient. In an embodiment, the proximal end 140 of the wire 132 can extend to a hub 120 of the catheter 102.

In use, the wire 132 can be in the retracted configuration as the distal tip 114 of the catheter 102 is advanced to a target location within the vasculature 80. The clinician can then manipulate the proximal end 140 of the wire 132 to slide the wire 132 longitudinally distally. The expandable portion 136 can abut against the abutment 116, disposed at the distal end 134 of the wire 132, transitioning the expandable portion 136 from the retracted configuration to the extended configuration. In the extended configuration, the apex 138 can impinge on a wall of the vessel 80 to position the catheter tip 114 in a spaced apart relationship relative to the wall of the vessel 80. In an embodiment, the apex 138 can impinge on a wall of the vessel 80 to center the catheter tip 114 within the vessel 80. In an embodiment, the apex 138 can impinge on a wall of the vessel 80 expand or distend the vessel to a larger diameter and provide a fluid pathway between the vessel wall and the distal tip 114 of the catheter 102.

In an embodiment, the centering device 130 can include two or more wires 132 disposed radially about the central axis 70. In an embodiment, as shown in FIGS. 2A-2B, the centering device 130 can include a first wire 132A disposed on a first side of the catheter 110 and a second wire 132B disposed on a second side of the catheter 110, opposite the first wire 132A across the central axis 70. In an embodiment, the first wire 132A and the second wire 132B in the extended configuration can distend the vessel along a first axis perpendicular to the longitudinal axis, e.g. the transverse axis. The diameter of the vessel along the opposite perpendicular axis, e.g. the lateral axis, can reduce to allow the transverse axis to increase to define a fluid path past the distal tip 114. In an embodiment, the centering device 130 can include three or more wires disposed radially equally or unequally about the central axis 70 (e.g. FIGS. 1B, 3B-3D) and configured to transition to the extended configuration and co-operate to center the central axis 70 of the catheter lumen 112 within a diametric center of the vasculature 80. In an embodiment, the centering device 130 can include three or more wires configured to transition to the extended configuration and expand or distend the vessel 80, increasing a fluid path between the vessel wall and the catheter tip 114 and reducing arterial insufficiency.

In an embodiment, as shown in FIGS. 2B-2C, the proximal end 140 of the wire 132 can be manipulated between one or more positions. For example, a first distal position can transition a first expandable portion 136A from the retracted configuration to the extended configuration (FIG. 2B). A second distal position, can transition a second expandable portion 136B from the retracted configuration to the extended configuration (FIG. 2C). Advantageously, the clinician can selectively expand one or more expandable portions 136 to widen a greater longitudinal portion of the vasculature 80 and reduce a fluid resistance and/or arterial insufficiency.

In an embodiment, the centering device 130 can further include a wire loop 142, metal bar, handle, or similar structure configured to couple to the wire 132, e.g. a proximal end 140, and can facilitate grasping and manipulating the wire 132, as described herein. In an embodiment, the loop 142 can couple a first wire 132A with one or more second wires 132B. In an embodiment, the loop 142 can be disposed adjacent the hub 120. In an embodiment, the first wire 132A, the loop 142, and the second wire 132B can be formed integrally.

In an embodiment, as shown in FIGS. 3A-3B, the system 100 can include an actuator 150 functionally coupled to one or more wires 132. In an embodiment, the actuator 150 can be coupled with one or more gears, levers, biasing members, or similar mechanisms configured to slide the proximal portion of the wire 132 longitudinally, as described herein, when the actuator 150 is actuated. In an embodiment, the actuator 150 can be disposed on the hub 120, however it will be appreciated that the actuator 150 can be disposed on other parts of the system 100 without limitation. As shown, the actuator 150 can be a push button, however it will be appreciated that various types of actuator are also contemplated, including switches, dials, thumb wheels, sliders, or the like, and are considered to fall within the scope of the present invention.

In an embodiment, as shown in FIG. 3A, with the actuator 150 in the first position, the expandable portion 136 can be disposed in the retracted position. A clinician can then actuate the actuator 150, e.g. depress the push button, which can slide the wire 132 longitudinally causing the expandable portion 136 to transition from the retracted configuration to the extended configuration (FIG. 3B). In an embodiment, the actuator 150 can transition between two or more positions to provide a graduated transition between a retracted configuration and one or more extended configurations. In an embodiment, the actuator 150 can transition between two or more positions to transition two or more expandable portions 136 between a retracted configuration and an extended configuration. Advantageously, the graduated actuator 150 can allow the user to select an amount of expansion of the expandable portion 136.

In an exemplary method of use, a medical device centering system 100 is provided generally including a catheter 102 and a centering device 130, as disclosed herein. As noted, embodiments described herein can be used with any elongate medical device configured to access a vasculature of a patient. A distal portion of the system 100 can be advanced through an insertion site 92 and into a vasculature 80 of a patient. A proximal portion of the system 100 can remain external to the patient, i.e. proximal of the insertion site 92. A clinician can then slide a proximal portion of the wire 132 longitudinally distally. In an embodiment, a clinician can manipulate a loop 142, or similar structure to slide one or more wires 132 longitudinally distally. In an embodiment, a clinician can actuate an actuator 150, or similar mechanism to slide one or more wires 132 longitudinally distally.

A distal tip 134 of the wire 132 can extend to a distal portion of the catheter 102. In an embodiment, the distal tip 134 of the wire 132 can be fixedly coupled to the body 110 of the catheter 102. In an embodiment, the distal tip 134 of the wire 132 can abut against an abutment 116 of the catheter 102. As the proximal portion of the wire 132 is slid longitudinally relative to the catheter body 102, the distal tip 134 remains substantially longitudinally stationary. As such, the expandable portion 136 of the wire 132 can transition from the retracted configuration to an extended configuration. In the extended configuration, an apex 138 of the expandable portion abuts against a wall of the vasculature 80 to maintain the distal tip 114 of the catheter 102 in a spaced apart relationship from the wall of the vasculature preventing the catheter tip 114 from contacting the wall of the vessel. In an embodiment, the apex 138 can elastically deform between the linear retracted configuration and a non-linear extended configuration. In an embodiment, the apex 138 can include a hinge or a living hinge configured to allow the expandable portion 136 to flex at the apex 138.

Embodiments disclosed herein can be configured to maintain the tip 114 of the catheter 102 in a spaced apart relationship from the wall of the vasculature 80. In an embodiment, two or more wires 132 can co-operate to maintain the tip 114 of the catheter 102 in a substantially central position within vasculature. In an embodiment, the expandable portion 136 can stretch or widen a diameter of the vasculature to increase a cross-sectional area of the vessel and provide an increased fluid path between the vessel wall and the catheter tip 114. In an embodiment, the expandable portion 136 can be disposed adjacent the distal tip 114 of the catheter body 110 and can expand sufficiently so as to prevent the distal tip 114 from contacting the vessel wall, e.g. a portion of the vessel wall disposed substantially perpendicular to the distal tip 114. This can mitigate the arterial insufficiency caused by the presence of the catheter within the vessel.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A centering device configured to access a vasculature of a patient, comprising:
a catheter including an elongate body defining a lumen communicating with an opening disposed adjacent a distal tip thereof; and
a centering device transitionable between a retracted configuration and an extended configuration, the centering device in the extended configuration configured to distance a distal tip of the catheter from a wall of the vasculature, the centering device including a wire extending longitudinally along an outer surface of the catheter and slidably engaged therewith, the wire comprising:
a distal tip coupled to a distal portion of the catheter;
an expandable portion disposed proximate the distal tip of the catheter and configured to transition between the retracted configuration and the extended configuration; and
a proximal portion configured to remain external to the patient.

2. The centering device according to claim 1, wherein the wire includes one of a shape memory material or nitinol.

3. The centering device according to claim 1, wherein the expandable portion is elastically deformable from the retracted configuration to the extended configuration.

4. The centering device according to claim 1, wherein the expandable portion includes a hinge or a living hinge.

5. The centering device according to claim 1, wherein the proximal portion is slidable along a longitudinal axis between the retracted configuration and the extended configuration.

6. The centering device according to claim 1, wherein a portion of the wire is disposed within a groove defined in an out surface of the catheter.

7. The centering device according to claim 6, further including the expandable portion of the wire disposed within the groove when the wire is in the retracted configuration.

8. The centering device according to claim 6, further including the proximal portion of the wire disposed within the groove and slidably engaged therewith.

9. The centering device according to claim 6, wherein a radial depth of the groove is equal to a thickness of the wire.

10. The centering device according to claim 6, further including a bridge formed integrally with the catheter body and extending across the groove, perpendicular thereto, the bridge configured to retain a portion of the wire within the groove.

11. The centering device according to claim 1, wherein a portion of the wire extends through a wire lumen extending through a wall of the catheter.

12. The centering device according to claim 1, further includes a loop formed integrally with the catheter body and configured to receive a portion of the wire extending therethrough.

13. The centering device according to claim 12, wherein a lumen of the loop is angled relative to the longitudinal axis.

14. The centering device according to claim 1, further including an abutment formed integrally with the catheter, the distal tip of the wire configured to abut against the abutment and prevent further distal movement of the distal tip relative to the catheter.

15. The centering device according to claim 1, wherein the proximal portion of the wire is coupled to a second proximal portion of a second wire to form a loop, the loop configured to be advanced distally to transition one of the wire or the second wire between the retracted configuration and the extended configuration.

16. The centering device according to claim 1, further including a push-button actuating mechanism configured to transition the wire between the retracted configuration and the extended configuration.

17. The centering device according to claim 1, wherein an apex of the expandable portion in the extended configuration is disposed radially outward from a central longitudinal axis relative to the retracted configuration.

18. The centering device according to claim 1, wherein an apex is disposed a first distance from the distal tip, the first distance being less than three times the diameter of the lumen.

19. The centering device according to claim 1, wherein the distal tip of the wire is disposed proximal of the distal tip of the catheter.

20. The centering device according to claim 1, wherein the distal tip of the wire is disposed a second distance from the distal tip, the second distance being less than two times the diameter of the lumen.

21. The centering device according to claim 1, wherein the proximal portion is disposed adjacent a bifurcation of the catheter.

22. The centering device according to claim 1, wherein the expandable portion in the extended configuration is configured to distend a vessel wall to a larger diameter along a first axis extending perpendicular to a longitudinal axis.

23. The centering device according to claim 1, wherein the centering device in the extended configuration is configured to prevent a distal tip of the catheter from contacting a wall of the vasculature.

24. The centering device according to claim 1, further including a second expandable portion disposed axially relative to the expandable portion, the second expandable portion including a second apex contacting a vessel wall at a different longitudinal configuration from the first apex.

25. The centering device according to claim 1, wherein the lumen of the catheter is fluid locked between a hub disposed at a proximal end of the elongate body and a distal tip.

26-35. (canceled)
